(19) United States
(12) Patent Application Publication
(10) Pub. No.: US 2023/0147718 A1
(43) Pub. Date: May 11, 2023

(54) COMPOSITION FOR CANCER PREVENTION OR TREATMENT, CONTAINING, AS ACTIVE INGREDIENT, SALVIANOLIC ACID B OR TANSHINONE I

(71) Applicant: GJMEDI, Busan (KR)

(72) Inventors: Se Bok JANG, Busan (KR); Mi Suk JEONG, Busan (KR); Chang Woo HAN, Busan (KR)

(73) Assignee: GJMEDI, Busan (KR)

(21) Appl. No.: 17/917,571

(22) PCT Filed: Apr. 2, 2021
(86) PCT No.: PCT/KR2021/004138

§ 371 Date: Oct. 7, 2022

(30) Foreign Application Priority Data
Apr. 9, 2020 (KR) 10-2020-0043477
Apr. 9, 2020 (KR) 10-2020-0043478

(51) Int. Cl. A61K 31/343; A61P 35/00; A61P 1/18; A61P 1/00; A61P 11/00; A61K 31/58

(52) U.S. Cl. CPC A61K 31/343; A61P 35/00; A61P 1/18; A61P 1/00; A61P 11/00; A61K 31/58

(57) ABSTRACT

The present invention relates to a composition for cancer prevention or treatment, containing, as an active ingredient, salvianolic acid B or tanshinone I. Specifically, provided are a pharmaceutical composition and a health functional food composition for KRAS-mutated cancer prevention or treatment, the compositions containing, as an active ingredient, salvianolic acid B or tanshinone I. Compared to a conventional therapeutic agent, salvianolic acid B or tanshinone I strongly binds to KRAS mutants so as to have an improved ability to target cancer cells induced by KRAS mutations, and thus diseases that are refractory to treatment with existing therapeutic agents, such as cancer in which KRAS mutations are overexpressed, can be effectively treated.

### TECHNICAL FIELD

The present disclosure relates to a composition for preventing or treating cancer including salvianolic acid B or tanshinone I as an active ingredient, and specifically, a composition for preventing or treating KRAS-mutated cancer including salvianolic acid B or tanshinone I as an active ingredient.

### BACKGROUND ART

Danshen (Scientific name: Salvia miltiorrhiza), an herbaceous perennial plant belonging to the family Lamiaceae, is a medicinal herb often used in Oriental medicine. Danshen is known to promote blood circulation and help treat cardiovascular diseases, and has been widely used in traditional medicine to treat cardiovascular-related diseases and hyperlipidemia in China and other Asian countries for hundreds of years.

Components of Danshen may be classified into about 40 types of lipophilic compounds and about 50 types of hydrophilic compounds, wherein salvianolic acid, one of the major physiologically active ingredients, is a water-soluble compound and divided into salvianolic acid A (SAA) and salvianolic acid B (SAB), with reports to have hepatoprotective efficacy, neuroprotective effect, and anticancer effect. In addition, Danshen also has strong antioxidant effects owing to a polyphenolic structure.

In addition, tanshinone is a lipophilic abietane diterpene compound including cryptotanshinone (CT), tanshinone IIA (TIIA), tanshinone I (TI), dihydrotanshinone I (DH-TI), isotanshinone I, tanshinone IIB, methyltanshinone, and isocryptotanshinone I.

The genetic causal factors for cancer development are alterations in proto-oncogenes, oncogenes, and tumor suppressor genes. Proto-oncogenes are normal genes in cells with the potential to become oncogenes while all oncogenes encode proteins and function with the proteins. In most cases, oncogenes are found as factors in signaling pathways. Oncogenes are formed naturally from proto-oncogenes through point mutations or translocations, thereby creating an altered state of cells hiding the mutations. Cancer occurs from oncogenes and tumor suppressor cells through a multistep process involving multiple mutations.

RAS is a factor receptor having a tyrosine kinase function and is an oncogene involved in oncogenesis in vivo. RAS genes form a small complex gene family of three functional members. This gene family consists of three functional members including HRAS, KRAS, and NRAS.

Mutant forms of the RAS gene have the potential to make normal cells cancerous. Proteins generated from the RAS gene play important roles in cell division, cell differentiation, and apoptosis, but it remains unclear by what mechanisms an oncogenic RAS induces tumor growth and regulates conversion to maintain the tumor microenvironment as well as which specific metabolic pathways are essential for survival of RAS-mediated tumor.

Cancers characterized by RAS mutations are not easy to treat using known therapies, and are considered as a negative predictor for survival rate of cancer patients to often have a poor prognosis.

Accordingly, there is a need to develop a new therapeutic agent that is more effective and stable for cancer diseases induced by RAS gene mutation.

### DISCLOSURE OF THE INVENTION

### Technical Goals

An object of the present disclosure is to provide a pharmaceutical composition or a health functional food composition exhibiting outstanding therapeutic or ameliorating effects on RAS gene mutated cancer.

Another object of the present disclosure is to provide a reagent composition for suppressing expression of the mutation or inhibiting proliferation of cancer cells induced by the mutation.

Another object of the present disclosure is to provide a method of inhibiting the mutation or cancer cells induced by the mutation.

### Technical Solutions

In order to achieve the above object, the present disclosure provides a pharmaceutical composition for preventing or treating KRAS-mutated cancer, including salvianolic acid B or tanshinone I as an active ingredient

The present disclosure provides a health functional food composition for preventing or ameliorating KRAS-mutated cancer, including salvianolic acid B or tanshinone I as an active ingredient.

The present disclosure provides a reagent composition for suppressing expression of KRAS mutation, including salvianolic acid B or tanshinone I as an active ingredient.

The present disclosure provides a reagent composition for inhibiting proliferation of cancer cells induced by KRAS mutation, including salvianolic acid B or tanshinone I as an active ingredient.

The present disclosure provides a method of suppressing expression of KRAS mutation, including treating animals other than humans with salvianolic acid B or tanshinone I.

The present disclosure provides a method of inhibiting proliferation of cancer cells induced by KRAS mutation, including treating animals other than humans with salvianolic acid B or tanshinone I.

### Advantageous Effects

Salvianolic acid B or tanshinone I according to the present disclosure binds strongly to a KRAS mutant compared to conventional therapeutic agents with little or low binding affinity for RAS surface, such that the targeting ability against cancer cells induced by KRAS mutation is improved, thereby effectively treating KRAS mutation-overexpressed cancer diseases which are difficult to treat with conventional therapeutic agents.

Furthermore, by using salvianolic acid B or tanshinone I, which is a material derived from natural products, it is possible to cope with issues concerning side effects and stability that cannot be solved by new synthetic drugs for intractable cancer diseases.

### BRIEF DESCRIPTION OF DRAWINGS

FIG. 1 shows graphs of identifying the effect of salvianolic acid B on the cell viability of human cancer cell lines according to an experimental example of the present disclosure, each representing the cell viability of (A) a pancreatic cancer cell line AsPC-1, (B) a colon cancer cell line HCT116, and (C) a lung cancer cell line NCI-H460. The experiment was performed three times, and each value is the mean±SEM of three independent experiments.

FIG. 2 shows graphs of identifying the effect of tanshinone I on the cell viability of human cancer cell lines according to an experimental example of the present disclosure, each representing the cell viability of (A) a colon cancer cell line HCT116, (B) a pancreatic cancer cell line AsPC-1, and (C) a lung cancer cell line NCI-H460. The experiment was performed three times, and each value is the mean±SEM of three independent experiments.

FIG. 3 is a graph of identifying the inhibitory efficacy of salvianolic acid B on a KRAS mutant according to an experimental example of the present disclosure, wherein radioisotope-free GTP was used as a competitor, and the data are expressed as mean±SD. Statistical tests were performed using Student's t-test. *p<0.05 was compared to a control under [α-32P]-labeled GTP (second column).

FIG. 4 is a graph of identifying the inhibitory efficacy of tanshinone I on a KRAS mutant according to an experimental example of the present disclosure, wherein radioisotope-free GTP was used as a competitor, and the data are expressed as mean±SD. Statistical tests were performed using Student's t-test. *p<0.05 was compared to a control under [α-32P]-labeled GTP (second column).

FIG. 5 is a result of analyzing the binding affinity between KRAS G12V and salvianolic acid B according to an experimental example of the present disclosure.

FIG. 6 is a result of analyzing the binding affinity between KRAS G12V and tanshinone I according to an experimental example of the present disclosure.

FIG. 7 is a 3D structure prediction model of a salvianolic acid B complex according to an experimental example of the present disclosure, wherein (A) is a ribbon diagram for a complex of KRAS G12V (green) and salvianolic acid B (pink), (B) shows a 3D bond structure model of KRAS G12V and salvianolic acid B using molecular surface representations, and (C) is a molecular structure of salvianolic acid B.

FIG. 8 is a 3D structure prediction model of a complex of KRAS G12V and tanshinone I according to an experimental example of the present disclosure, wherein (A) is a ribbon diagram for a complex of KRAS G12V (green) and tanshinone I (cyan), (B) shows the 3D bond structure model of KRAS G12V and tanshinone I using molecular surface representations, and (C) shows a molecular structure of tanshinone I.

### BEST MODE FOR CARRYING OUT THE INVENTION

Hereinafter, the present disclosure will be described in more detail.

The present inventors completed the present disclosure by identifying that salvianolic acid B, one of salvianolic acids as a natural extract isolated from Danshen, and tanshinone I act as a new inhibitor capable of inhibiting RAS, a cancer-causing protein, through experiments such as cell viability analysis, guanine nucleotide binding assay, isothermal titration thermometry analysis, and 3D structure model prediction.

The term “prevention” as used herein refers to any action that suppresses or delays the onset of a cancer disease or at least one or more symptoms thereof, by administration of a pharmaceutical composition or health functional food composition according to the present disclosure. The prevention also includes treatment of a subject showing effectiveness in the disease to prevent or avoid recurrence.

The term “treatment” as used herein refers to any act of ameliorating or beneficially changing symptoms, such as alleviating, reducing, or eliminating a cancer disease or at least one or more symptoms thereof by administration of the pharmaceutical composition according to the present disclosure.

The term “amelioration” as used herein refers to any act of ameliorating or beneficially changing symptoms, such as alleviating, reducing, or eliminating a cancer disease or at least one or more symptoms thereof by ingestion of the health functional food composition according to the present disclosure.

The term “pharmaceutical composition” as used herein refers to a composition administered for a specific purpose, to prevent or treat a cancer disease or at least one or more symptoms thereof for the purpose of the present disclosure.

The term “health functional food” as used herein includes food manufactured and processed using raw materials or ingredients useful for human body in accordance with Act No. 6727 of Law for Health Functional Foods, and also refers to, in addition to nutritional supply, foods with high medical and therapeutic effects that are processed to efficiently derive bioregulatory functions such as prevention of cancer diseases, body defense, immunity, and recovery for the purpose of the present disclosure.

The present disclosure provides a pharmaceutical composition for preventing or treating KRAS-mutated cancer, including salvianolic acid B or tanshinone I as an active ingredient.

As described above, the salvianolic acid B or tanshinone I, one of the major physiologically active ingredients of Danshen, may be directly isolated or extracted from natural products by methods well known in the art or prepared by chemical synthesis and used by selecting one that is commercially available, but the method or material is not particularly limited.

The salvianolic acid B or tanshinone I may be used in the form of a pharmaceutically or sitologically acceptable salt within the range having the same efficacy.

The term “pharmaceutically or sitologically acceptable” as used herein refers to a state with no toxicity to cells or humans exposed to the composition.

The salt may be used in the form of any one of pharmaceutically or sitologically acceptable base salts or acid salts. The basic salt may be used in the form of any one of an organic base salt and an inorganic base salt and selected from the group consisting of a sodium salt, a potassium salt, a calcium salt, a lithium salt, a magnesium salt, a cesium salt, an aminium salt, an ammonium salt, a triethylaminium salt, and pyridinium salt.

As the acid salt, an acid addition salt formed by a free acid is useful. As the free acid, an inorganic acid and an organic acid may be used, wherein hydrochloric acid, hydrobromic acid, sulfuric acid, sulfurous acid, phosphoric acid, double phosphoric acid, and nitric acid may be used as the inorganic acid while citric acid, acetic acid, maleic acid, malic acid, fumaric acid, gluconic acid, methanesulfonic acid, benzenesulfonic acid, camphorsulfonic acid, oxalic acid, malonic acid, glutaric acid, acetic acid, glycolic acid, succinic acid, tartaric acid, 4-toluenesulfonic acid, galacturonic acid, embonic acid, glutamic acid, citric acid, aspartic acid, and stearic acid may be used as the organic acid, but are not limited thereto, and salts that are formed with various inorganic and organic acids commonly used in the art may be included.

In addition, the salvianolic acid B or tanshinone I may include pharmaceutically or sitologically acceptable salts as well as all salts, hydrates, solvates, and derivatives that may be prepared by conventional methods. The addition salt may be prepared by a conventional method and prepared by addition of an excess organic base after dissolving in a water-miscible organic solvent such as acetone, methanol, ethanol, or acetonitrile, or by precipitation or crystallization after adding an aqueous base solution of an inorganic base. Alternatively, the addition salt may be obtained by drying after evaporation of the solvent or excess base from the mixture, or by suction filtration of the precipitated salt.

In the pharmaceutical composition according to the present disclosure, the salvianolic acid B or tanshinone I may be bound to a KRAS mutant.

As described above, the KRAS is a subtype of the oncogene RAS involved in tumorigenesis, and the KRAS mutation may be a mutation at a position of G12 amino acid, preferably a G12V mutation.

According to an experimental example of the present disclosure, the salvianolic acid B or tanshinone I exhibited a strong binding affinity to directly bind to the surface of KRAS G12V protein of which some amino acid residues were stabilized by forming hydrogen bonds with salvianolic acid B or tanshinone I.

In the pharmaceutical composition according to the present disclosure, the salvianolic acid B or tanshinone I may inhibit proliferation of cancer cells induced by KRAS mutation and thus effectively prevent or treat cancer induced by KRAS mutation, preferably a cancer selected from the group consisting of pancreatic cancer, colorectal cancer, and lung cancer with KRAS mutation overexpressed, more preferably pancreatic cancer.

The KRAS mutation is in the most aggressive and lethal cancer pathway while the mutation is found by 25% in lung cancer, 52% in colorectal cancer, and 90% in pancreatic cancer, and in some patients with advanced cancer, the mutation causes premature death.

The pharmaceutical composition according to the present disclosure may be prepared according to a conventional method in the pharmaceutical field. The pharmaceutical composition may be combined with an appropriate pharmaceutically acceptable carrier depending on the formulation, and if necessary, may be prepared by further including excipients, diluents, dispersants, emulsifiers, buffers, stabilizers, binders, disintegrants, and solvents. The appropriate carrier and the like do not inhibit the activity and properties of salvianolic acid B or tanshinone I according to the present disclosure, and may be selected differently depending on the dosage type and formulation.

The pharmaceutical composition according to the present disclosure may be applied in any formulation, and more specifically, it may be used by being formulated into oral dosage forms, external preparations, suppositories, and parenteral dosage forms of sterile injection solutions according to conventional methods.

Of the oral dosage forms, solid formulation may be prepared by mixing, in the form of tablets, pills, powders, granules, and capsules, at least one or more excipients, for example, starch, calcium carbonate, sucrose, lactose, sorbitol, mannitol, cellulose, and gelatin, and lubricants such as magnesium stearate and talc may be included in addition to simple excipients. In addition, the capsule formulation may further include a liquid carrier such as fatty oil in addition to the above-mentioned substances.

Of the oral dosage forms, liquid formulations may include suspensions, solutions, emulsions, and syrups, and in addition to water and liquid paraffin that are commonly used simple diluents, various excipients such as wetting agents, sweeteners, fragrances, and preservatives may be included.

The parenteral formulation may include a sterile aqueous solution, a non-aqueous solution, a suspension, an emulsion, a freeze-dried formulation, and a suppository. As the non-aqueous solvent and suspension, propylene glycol, polyethylene glycol, vegetable oils such as olive oil, and injectable esters such as ethyl oleate may be used. As the base of the suppository, witepsol, macrogol, Tween 61, cacao butter, laurin fat, glycerogelatin, and the like may be used. Not limited thereto, any suitable agent known in the art may be used.

In addition, the pharmaceutical composition according to the present disclosure may further be added with calcium or vitamins to enhance therapeutic efficacy.

In the pharmaceutical composition according to the present disclosure, the pharmaceutical composition may be administered in a pharmaceutically effective amount. The term “pharmaceutically effective amount” as used herein refers to an amount that is sufficient to treat a disease at a reasonable benefit/risk ratio applicable to medical treatment without causing side effects.

The effective dose level of the pharmaceutical composition may vary depending on the purpose of use, the age, sex, weight and health status of a patient, the type of disease, severity, drug activity, sensitivity to drug, administration method, administration time, administration route and excretion rate, treatment duration, factors including drugs that are blended or used in combination with, and other factors well known in the medical field. For example, although not constant, generally 0.001 to 100 mg/kg, preferably 0.01 to 10 mg/kg, may be administered once to several times a day. The above dosage does not limit the scope of the present disclosure in any way.

The pharmaceutical composition according to the present disclosure may be administered to any animal in which cancer may be developed, and the animal may include, for example, not only humans and primates, but also livestock such as cattle, pigs, horses, and dogs.

The pharmaceutical composition according to the present disclosure may be administered by an appropriate administration route according to the type of the formulation, and may be administered through various routes, either oral or parenteral, as long as it may reach the target tissue. The administration may be performed via conventional methods with no particular limitation, including, for example, oral, rectal or intravenous, muscle, skin application, respiratory inhalation, and intrauterine dural or intracerebroventricular injection.

The pharmaceutical composition according to the present disclosure may be used alone for prevention or treatment of cancer or in combination with surgery or other drug treatment.

The present disclosure provides a health functional food composition for preventing or ameliorating KRAS-mutated cancer, including salvianolic acid B or tanshinone I as an active ingredient.

The salvianolic acid B or tanshinone I may bind to a KRAS mutant and inhibit proliferation of cancer cells induced by KRAS mutation, such that it may be applied as a health functional food composition for prevention or amelioration of KRAS-mutated cancer.

Corresponding features may be substituted for the above-mentioned parts.

In the health functional food composition according to the present disclosure, the health functional food may be prepared as powder, granules, tablets, capsules, syrups or beverages for the purpose of preventing or ameliorating KRAS-mutated cancer. There is no limitation in the form that the health functional food may take, and it may be formulated in the same manner as the pharmaceutical composition to be used as a functional food or added to various foods.

In the health functional food composition according to the present disclosure, the health functional food may include all foods in a conventional sense. For example, beverages and various drinks, fruits and processed foods thereof (canned fruit, jam, etc.), fish, meat and processed foods thereof (ham, bacon, etc.), breads and noodles, cookies and snacks, and dairy products (butter, cheese, etc.) may be possible, and functional foods may all be included in a conventional sense. It may also include food used as feed for animals.

The health functional food composition according to the present disclosure may be prepared by further including a sitologically acceptable food additive and other suitable supplementary components commonly used in the art. Unless otherwise specified, the suitability as a food additive may be determined according to the standards and criteria for the relevant item in accordance with the general rules and general test methods of the Korean Food Additives Codex approved by the Ministry of Food and Drug Safety. The items listed in the ‘Korean Food Additives Codex’ include, for example, chemical compounds such as ketones, glycine, calcium citrate, nicotinic acid, and cinnamic acid; natural additives such as persimmon pigment, licorice extract, crystalline cellulose, Kaoliang color, and guar gum; and mixed preparations such as a sodium L-glutamate preparation, a noodle-added alkali agent, a preservative preparation, and a tar dye.

The other supplementary components may further include, for example, flavoring agents, natural carbohydrates, sweeteners, vitamins, electrolytes, colorants, pectic acid, alginic acid, organic acids, protective colloidal thickeners, pH adjusters, stabilizers, preservatives, glycerin, alcohols, and carbonation agents. In particular, as the natural carbohydrate, monosaccharides such as glucose and fructose, disaccharides such as maltose and sucrose, polysaccharides such as dextrin and cyclodextrin, and sugar alcohols such as xylitol, sorbitol, and erythritol may be used, and as the sweetener, natural sweeteners such as taumatine and stevia extract or synthetic sweeteners such as saccharin and aspartame may be used.

The effective dose of the salvianolic acid B or tanshinone I included in the health functional food according to the present disclosure may be appropriately adjusted according to the purpose of use, such as prevention or amelioration of KRAS-mutated cancer.

The health functional food composition causes no side effects that may occur during long-term administration of general drugs by using food as a raw material, and may be taken as a supplement for preventing or ameliorating cancer owing to excellent portability.

The present disclosure provides a reagent composition for suppressing expression of KRAS mutation, including salvianolic acid B or tanshinone I as an active ingredient.

Preferably, it may be for suppression of expression of KRAS mutant KRAS G12V.

The present disclosure provides a reagent composition for inhibiting proliferation of cancer cells induced by KRAS mutation, including salvianolic acid B or tanshinone I as an active ingredient.

Preferably, it may be a pancreatic cancer, colorectal cancer, or lung cancer cell in which the KRAS mutation is overexpressed, and more preferably a pancreatic cancer cell.

Corresponding features may be substituted for the above-mentioned parts.

In addition, the present disclosure provides a method of suppressing expression of KRAS mutation, including treating animals other than humans with salvianolic acid B or tanshinone I.

The present disclosure provides a method of inhibiting proliferation of cancer cells induced by KRAS mutation, including treating an animal other than humans with salvianolic acid B or tanshinone I.

Corresponding features may be substituted for the above-mentioned parts.

### Modes for Carrying Out the Invention

Hereinafter, to help the understanding of the present disclosure, example embodiments will be described in detail. However, the following example embodiments are merely illustrative of the contents of the present disclosure, and the scope of the present disclosure is not limited to the following examples. The example embodiments of the present disclosure are provided to more completely explain the present disclosure to those of ordinary skill in the art.

<Experimental Example 1> Cell Viability Analysis

1. Experimental Method

To verify the efficacy of salvianolic acid B (Sigma-Aldrich) and tanshinone I (Sigma-Aldrich) on tumor cells, MTT (3-(4,5-dimethylthiazol-2-yl)-2,5-diphenyltetrazolium bromide) assay was applied to measure cell viability.

For the experiment, AsPC-1 (pancreatic cancer), HCT116 (colon cancer), and NCI-H460 (lung cancer) were used as tumor cell lines, and each tumor cell was dispensed by 1×10³ cells/well (100 μL). After dispensing, all cells were attached to the wells for 24 hours. Before treatment of salvianolic acid B or tanshinone I, media of tumor cells were replaced with fresh media (RPMI-1640) and treated with salvianolic acid B or tanshinone I with increasing concentration gradients. After culturing the drug-treated tumor cells for 24 and 48 hours, MTT at a concentration of 5 mg/mL was added to each tumor cell, followed by culture for 90 minutes again. Finally, the optical density (OD) of the tumor cells was measured at 540 nm using a microplate reader (Molecular Devices, USA). The analysis was performed three or more times.

2. Experiment Result

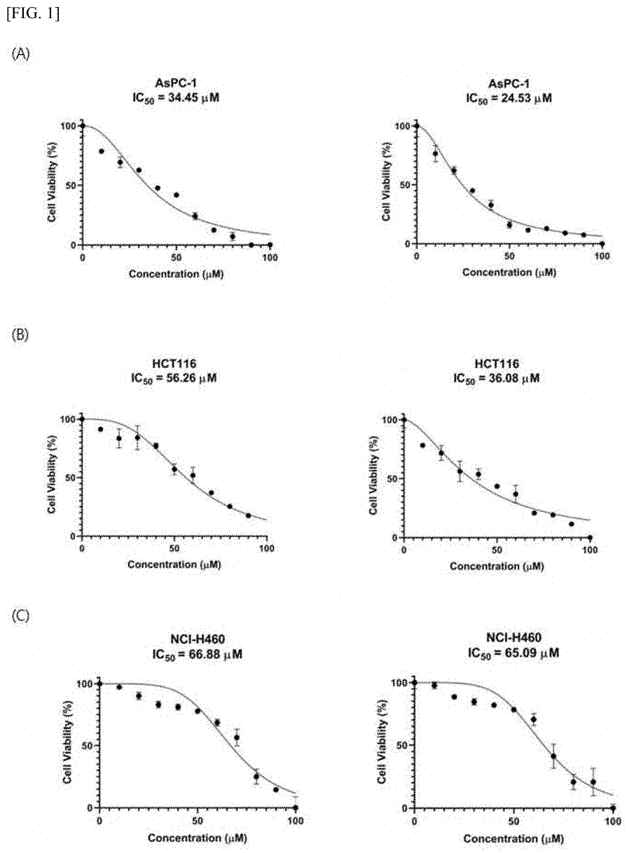

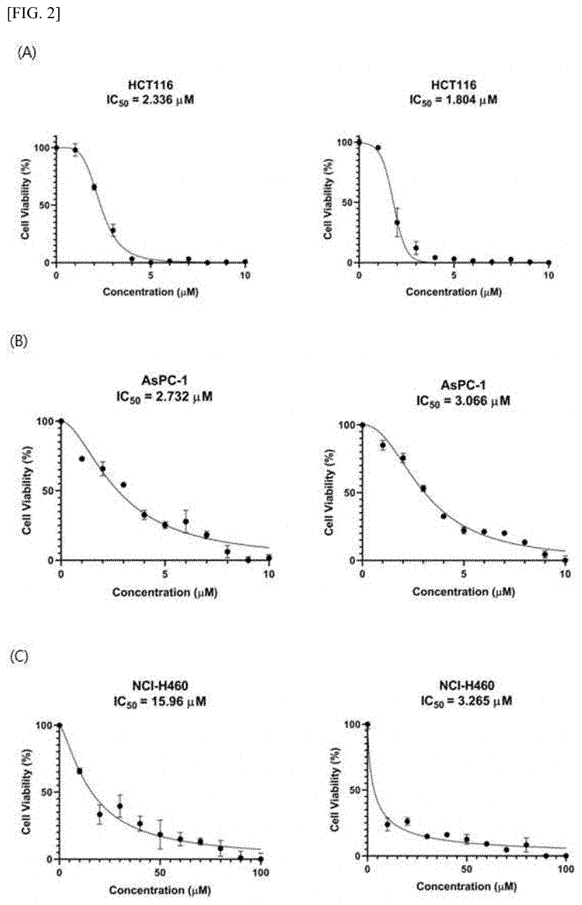

The inhibitory effect of salvianolic acid B or tanshinone I on tumor cell proliferation was tested using three different tumor cell lines. As a result, as shown in FIGS. 1 and 2, when salvianolic acid B or tanshinone I was cultured with cells for 24 and 48 hours, respectively, a decrease in cell viability was observed in AsPC-1 (pancreatic cancer), HCT116 (colon cancer), and NCI-H460 (lung cancer) cell lines. Salvianolic acid B or tanshinone I showed very superior inhibitory effects on AsPC-1, HCT116, and NCI-H460 in a time- and dose-dependent manner. These three tumor cell lines showed different sensitivities to salvianolic acid B or tanshinone I, respectively.

<Experimental Example 2> Guanine Nucleotide Binding Assay

1. Experimental Method

The recombinant gene KRAS G12V was inserted into E. coli BL21 (DE3) for expression. The overexpressed protein was purified using affinity chromatography and gel filtration chromatography. Purified KRAS G12V protein was bound to Ni-NTA beads using binding buffer [50 mM Hepes (pH 7.5), 100 mM NaCl, 2 mM MgCl₂, 1 mM EDTA, and 1 mM DTT] at 4° C. Ni-NTA beads bound with KRAS G12V proteins were washed with binding buffer and then reacted with radioisotope [α-³²P] GTP (2,500 cpm/pmol), GTP, and salvianolic acid B or tanshinone I at 37° C. The beads were then washed with wash buffer [20 mM Tris-HCl (pH 7.4), 100 mM NaCl, and 2 mM MgCl₂]. 200 mM imidazole was used to elute the bead-bound KRAS G12V protein, and radioactive nucleotides were quantified by liquid scintillation counting.

2. Experiment Result

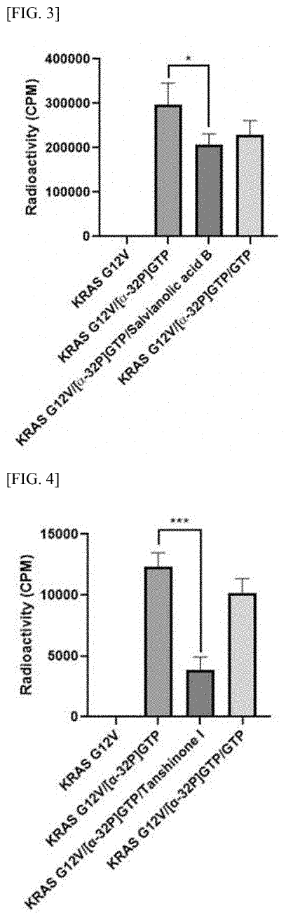

The binding ability of salvianolic acid B or tanshinone I to the KRAS cancer-inducing mutant G12V was identified using guanine nucleotide binding assay. As a result of the analysis, it was found that the activity of radioisotope [α-³²P] GTP binding of KRAS G12V was decreased in the presence of salvianolic acid B or tanshinone I as shown in FIGS. 3 and 4.

<Experimental Example 3> Isothermal Titration Thermometric Analysis

1. Experimental Method

In order to identify the affinity of salvianolic acid B or tanshinone I for the KRAS mutant KRAS G12V protein, isothermal titration calorimetry (ITC) was used. KRAS G12V protein was dialyzed using buffer [20 mM Tris-HCl (pH 7.4), 100 mM NaCl, and 2 mM MgCl₂]. Next, salvianolic acid B or tanshinone I was dissolved using the same buffer, and then the prepared KRAS G12V protein and salvianolic acid B or tanshinone I were injected into an isothermal calorimeter to perform isothermal titration thermometry. Titration measurement was performed at 25° C., involved with 20 injections at an interval of 200 seconds while salvianolic acid B or tanshinone I was stirred at 1,000 rpm. ΔS was calculated from standard thermodynamic equations using the calculated K and ΔH values. Isothermal titration thermometric analysis was performed using a MicroCal AutoITC200 (GE Healthcare, Sweden), and data were analyzed using the Origin 7.0 program.

2. Experiment Result

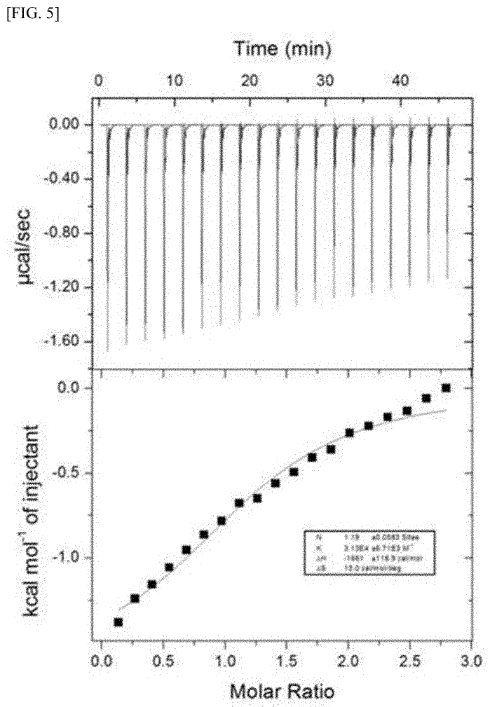

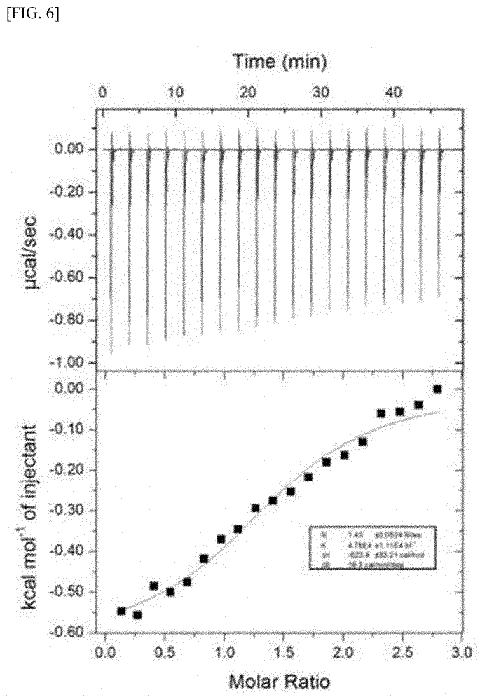

As shown in FIGS. 5 and 6, the binding affinity between the KRAS G12V mutant protein and salvianolic acid B or tanshinone I was identified using ITC analysis, and ΔS was calculated from the standard thermodynamic equation using the calculated K and ΔH values. Based on the equation, the binding affinity of salvianolic acid B to KRAS G12V was K_{D}=32 μM, and that of tanshinone I to KRAS G12V was K_{D}=21 μM.

<Experimental Example 4> Prediction of a 3D Structure Model

1. Experimental Method

To investigate the binding between the KRAS mutant KRAS G12V protein and salvianolic acid B or tanshinone I, a complex model was predicted by a 3D structure. For prediction, analysis was performed using the KRAS G12V model of the protein database and a salvianolic acid B or tanshinone I model of Pubchem.

2. Experiment Result

In order to understand the binding between the KRAS mutant KRAS G12V protein and salvianolic acid B or tanshinone I, the 3D structure of these complexes was predicted by molecular modeling.

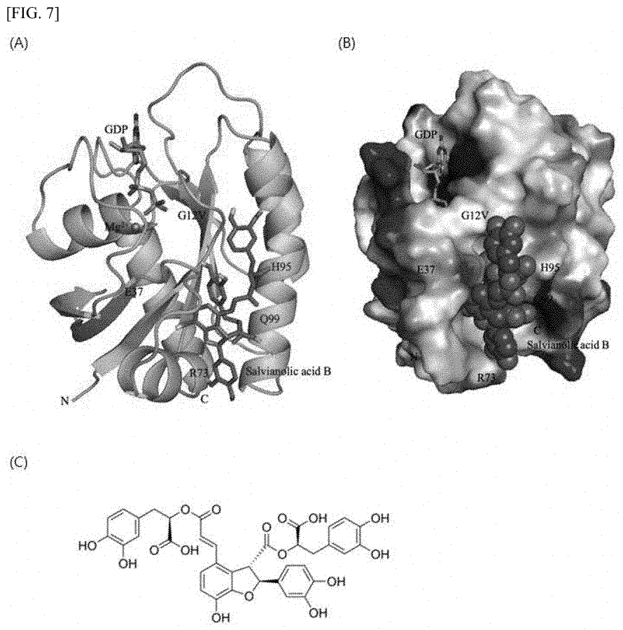

As a result of the prediction, as shown in FIG. 7, it was found that the binding affinity of the KRAS mutant KRAS G12V and salvianolic acid B was −5.4 kcal/mol, and 5 amino acid residues (G12V, E37, R73, H95, and Q99) of the KRAS G12V protein were stabilized by forming hydrogen bonds with salvianolic acid B.

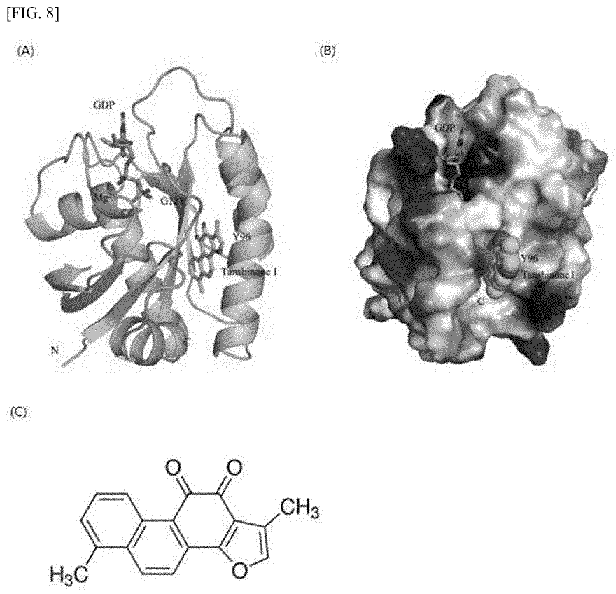

In addition, as shown in FIG. 8, it was found that the binding affinity between the KRAS mutant KRAS G12V and tanshinone I was −6.3 kcal/mol, and one amino acid residue (Y96) of the KRAS G12V protein was stabilized by forming hydrogen bonds with tanshinone I.

As described above, a specific part of the content of the present disclosure is described in detail, for those of ordinary skill in the art, it is clear that the specific description is only a preferred embodiment, and the scope of the present disclosure is not limited thereby. Accordingly, the substantial scope of the present disclosure may be defined by the appended claims and their equivalents.

1. A method of preventing or treating KRAS-mutated cancer in a subject in need thereof, comprising:
administering a pharmaceutical composition comprising salvianolic acid B or tanshinone I as an active ingredient to the subject.

2. The method of claim 1, wherein the salvianolic acid B or tanshinone I binds to a KRAS mutant.

3. The method of claim 1, wherein the salvianolic acid B or tanshinone I inhibits proliferation of cancer cells induced by KRAS mutation.

4. The method of claim 1, wherein the KRAS-mutated cancer is selected from the group consisting of pancreatic cancer, colorectal cancer, and lung cancer with KRAS mutation overexpressed.

5. The method of claim 1, wherein the KRAS mutation is G12V mutation.

6-8. (canceled)

9. A method of suppressing expression of KRAS mutation, comprising treating an animal other than humans with salvianolic acid B or tanshinone I.

10. A method of inhibiting proliferation of cancer cells induced by KRAS mutation, comprising treating an animal other than humans with salvianolic acid B or tanshinone I.
